(19) United States
(12) Patent Application Publication
(10) Pub. No.: US 2017/0296326 A1
(43) Pub. Date: Oct. 19, 2017

(54) STENT GRAFT DEVICE

(71) Applicant: National University of Singapore, Singapore (SG)

(72) Inventors: Soo Yeng Benjamin CHUA, Singapore (SG); Tsui Ying Rachel HONG, Singapore (SG); Tswen Wen Victor LEE, Singapore (SG); Siang Lin YEOW, Singapore (SG); Wee Chuan Melvin LOH, Singapore (SG)

(21) Appl. No.: 15/513,316

(22) PCT Filed: Sep. 22, 2015
(86) PCT No.: PCT/SG2015/050333

§ 371 Date: Mar. 22, 2017

Related U.S. Application Data
(60) Provisional application No. 62/053,758, filed on Sep. 22, 2014.

(51) Int. Cl. A61F 2/07; A61F 2/06

(52) U.S. Cl. CPC A61F 2/07; A61F 2250/0023; A61F 2002/068; A61F 2210/0076; A61F 2230/0026

(57) ABSTRACT

The present disclosure relates to stent graft devices for endovascular repair of aneurysms. A stent graft device according to the present disclosure comprises: a membrane defining a lumen between a proximal end and a distal end of the membrane, the lumen for fluid communication distally therethrough; a plurality of fenestrations disposed on the membrane and fluidly communicable with the lumen; and a plurality of protrusions carried by the membrane, each protrusion extending inwardly into or outwardly from the lumen. Fluid communicated from the plurality of fenestrations is deflectable or deflected by the plurality of protrusions.

### TECHNICAL FIELD

The present disclosure generally relates to stent graft devices. More particularly, aspects of the present disclosure are directed to stent graft devices for endovascular repair of aneurysms that can occur within any blood vessel in human or animal bodies.

### BACKGROUND

An aneurysm (e.g. saccular aneurysm, fusiform aneurysm, and dissecting aneurysm) is a localized dilatation in the wall of a blood vessel, e.g. an artery or vein, due to the weakening of the arterial wall structure. Common regions susceptible to the formation of aneurysms include the cerebral arteries (e.g. internal carotid artery, middle cerebral artery, anterior cerebral artery, and posterior cerebral artery), carotid arteries (e.g. common carotid artery and external carotid artery), peripheral arteries (e.g. iliac artery, popliteal artery, femoral artery), and aorta (e.g. aortic arch, thoracic aorta, thoracoabdominal aorta, and abdominal aorta).

Major segments of an affected aorta include the ascending aorta, aortic arch, descending aorta, thoracic aorta, thoracoabdominal aorta, abdominal aorta, and infrarenal aorta. FIG. 1 illustrates a location for a thoracic aortic aneurysm and a location for an abdominal aortic aneurysm. Up to 45% of aortic aneurysms can involve the extracranial cerebral blood vessels (e.g. aortic arch aneurysms), the abdominal visceral vessels (thoracoabdominal aneurysms), or the renal arteries (pararenal/juxtarenal aortic aneurysm). These aneurysms are particularly difficult to treat as surgery must focus on preserving flow to these cerebral vessels or visceral vessels. FIG. 2 illustrates the intracranial vasculature and shows the most frequent locations for cerebral aneurysms.

The prevalence of aortic aneurysms in the developed world is high. It has been estimated that there are around 4.5 million cases of aortic aneurysms in the developed world with an incidence rate of 600,000 cases each year. Abdominal aortic aneurysms have an estimated prevalence of 1.3% to 12.7% depending on the age group. The incidence and prevalence of abdominal aortic aneurysms increases with age and is three times more common in men than in women with mortality rates of ruptured aneurysms at 80% to 90%. The global market for endovascular repair of abdominal aortic aneurysms was valued at about USD 820 million in 2008 and may be expected to expand and/or grow on a worldwide basis to approximately USD 1.6 billion by 2015.

Current modalities for treatment include using aortic stent grafts to reline the aneurysmal portion of the aorta, thereby excluding the aneurysm from blood circulation and causing thrombosis in the aneurysm sac. One of the key limitations for currently known aortic stent grafts is the development of endoleaks post insertion or implantation. There are four types of endoleaks.
Type 1—Leak around the proximal and distal ends of the aortic stent graft.
Type 2—Back bleeding from a covered vessel (e.g. lumbar arteries) that comes off from the aneurysm sac.
Type 3—Leak around modular components of aortic stent grafts.
Type 4—Leak through a porous aortic stent graft wall or fabric.

As such, patients being considered for treatment with currently known aortic stent grafts have to meet certain anatomical criteria. In particular, to prevent Type 1 endoleaks, there should be an adequate seal zone for the proximal end of the aortic stent graft. This translates clinically as a need and/or requirement for a minimum of a 15 mm length/distance of good quality aorta from the nearest visceral artery to act as a good seal zone.

Currently known non-customized aortic stent grafts are often made of non-porous materials, such as polyethylene terephthalate (Dacron) and polytetrafluoroethylene (PTFE). The aortic stent grafts cannot be laid across visceral vessels as this would result in obliteration or obstruction of blood supply to the viscera with irreversible and often fatal consequences. As such, in view of the above, currently known standard off-the-shelf aortic stent grafts cannot be effectively used for patients with aortic arch aneurysms, thoracoabdominal aortic aneurysms, or juxtarenal aortic aneurysms as the potential for endoleaks developing is undesirably high.

Currently, aortic stent grafts can also be tailor-made for individual patients. Customized aortic stent grafts may have individual fenestrations (i.e. holes) to fit or match the visceral arteries. However, these customized aortic stent grafts require intensive planning and design, take a long time to manufacture, and are expensive. As a result of these disadvantages, customized aortic stent grafts are not suitable for use in emergent and/or emergency situations, for example in the case of ruptured or leaking aneurysms.

Therefore, in order to address or alleviate at least one of the aforementioned problems and/or disadvantages, there is a need to provide stent graft devices in which there are at least improved feature(s) over the prior art.

### SUMMARY

According to a first aspect of the present disclosure, there is a stent graft device comprising: a membrane defining a lumen between a proximal end and a distal end of the membrane, the lumen for fluid communication distally therethrough; a plurality of fenestrations disposed on the membrane and fluidly communicable with the lumen; and a plurality of protrusions carried by the membrane, each protrusion extending inwardly into or outwardly from the lumen. Fluid communicated from the plurality of fenestrations is deflectable or deflected by the plurality of protrusions.

According to a second aspect of the present disclosure, there is an endovascular repair kit comprising: a stent delivery system; and a stent graft device. The stent graft device comprises: a membrane defining a lumen between a proximal end and a distal end of the membrane, the lumen for fluid communication distally therethrough; a stent coaxially disposed within the lumen; a plurality of fenestrations disposed on the membrane and fluidly communicable with the lumen; and a plurality of protrusions carried by the membrane, each protrusion extending inwardly into or outwardly from the lumen. Fluid communicated from the plurality of fenestrations is deflectable or deflected by the plurality of protrusions.

The deflection of fluid communication away from its usual direction toward the distal end adjusts the blood flow dynamics relative to an aneurysm sac. The implantation of the stent graft device in an artery can affect blood flow into the aneurysm sac by substantially reducing the fluid distal flow velocity in the aneurysm sac. The lower fluid flow velocity in the distal direction reduces the fluid pressure at the distal side of the aneurysm sac, thereby mitigating the risks of further expansion and/or rupture of the aneurysm sac, which is caused due to extensive blood flow into the aneurysm sac. Advantageously, the stent graft device facilitates localized thrombosis in the aneurysm sac 202 which can lead to relining of the arterial wall of that particular vessel segment, and subsequently to the occlusion and regression of the aneurysm sac.

Stent graft devices according to the present disclosure are thus disclosed hereinabove. Various features, aspects, and advantages of the present disclosure will become more apparent from the following detailed description of the embodiments of the present disclosure, by way of non-limiting examples only, along with the accompanying drawings in which like numerals represent like components.

### BRIEF DESCRIPTION OF THE DRAWINGS

FIG. 1 is an illustration of typical locations of a thoracic aortic aneurysm and an abdominal aortic aneurysm in a human body.

FIG. 2 is an illustration of an intracranial vasculature and the most frequent locations of cerebral aneurysms.

FIG. 3A is an illustration of an aortic aneurysm before implantation of a stent graft device according to an embodiment of the present disclosure.

FIG. 3B is an illustration of the aortic aneurysm of FIG. 3A, immediately after implantation of the stent graft device according to an embodiment of the present disclosure.

FIG. 3C is an illustration of the aortic aneurysm of FIG. 3B, after implantation of the stent graft device according to an embodiment of the present disclosure.

FIG. 4A is an illustration of a saccular aneurysm before implantation of a stent graft device according to an embodiment of the present disclosure.

FIG. 4B is an illustration of a saccular aneurysm of FIG. 4A immediately after implantation of the stent graft device according to an embodiment of the present disclosure.

FIG. 4C is an illustration of a saccular aneurysm of FIG. 4A, after implantation of the stent graft device according to an embodiment of the present disclosure.

FIG. 5 is an illustration of the positioning of a stent graft device across a carotid artery, according to an embodiment of the present disclosure.

FIG. 6A is an isometric illustration of a stent graft device for flow diversion/fluid deflection, according to an embodiment of the present disclosure.

FIG. 6B is a top/planar illustration of the stent graft device of FIG. 6A, according to an embodiment of the present disclosure.

FIG. 7A to FIG. 7C are illustrations of a protrusion and fenestration of a stent graft device, according to an embodiment of the present disclosure.

FIG. 8A to FIG. 8C are illustrations of shapes of protrusions of a stent graft device, according to an embodiment of the present disclosure.

FIG. 9A to FIG. 9C are illustrations of positions and/or arrangements of protrusions of a stent graft device, according to an embodiment of the present disclosure.

FIG. 10A to FIG. 10C are additional illustrations of positions and/or arrangements of protrusions of a stent graft device, according to an embodiment of the present disclosure.

FIG. 11A and FIG. 11B are illustrations of a stent graft device for flow diversion/fluid deflection, according to an embodiment of the present disclosure.

FIG. 12A to FIG. 12C are illustrations of stimulated fluid/blood flow dynamics around an aneurysm sac, according to an embodiment of the present disclosure.

FIG. 12D is an illustration of the stent graft device of FIG. 11A implanted in an artery with a fusiform aneurysm, according to an embodiment of the present disclosure.

FIG. 13A is an illustration a stent graft device with adjustable porosity, according to an embodiment of the present disclosure.

FIG. 13B is an illustration of a portion of the stent graft device of FIG. 13A with separated membranes, according to an embodiment of the present disclosure.

FIG. 13C is an illustration of the stent graft device of FIG. 13A adjusted to minimum porosity and maximum coverage.

FIG. 13D is an illustration of the stent graft device of FIG. 13A adjusted to maximum porosity and minimum coverage.

### DETAILED DESCRIPTION

In the present disclosure, depiction of a given element or consideration or use of a particular element number in a particular FIG. or a reference thereto in corresponding descriptive material can encompass the same, an equivalent, or an analogous element or element number identified in another FIG. or descriptive material associated therewith. The use of “/” in a FIG. or associated text is understood to mean “and/or” unless otherwise indicated. The recitation of a particular numerical value or value range herein is understood to include or be a recitation of an approximate numerical value or value range, for instance, within +/−20%, +/−15%, +/−10%, +/−5%, or +/−0%. With respect to recitations herein directed to dimensional or numerical comparisons or equivalence, reference to the terms “generally,” “approximately,” or “substantially” is understood as falling within +/−20%, +/−15%, +/−10%, +/−5%, or +/−0% of a representative/example comparison, or a specified or target value or value range; and reference to the term “essentially” is understood as falling within +/−10%, +/−5%, +/−2%, +/−1%, or +/−0% of a representative/example comparison, or a specified or target value or value range.

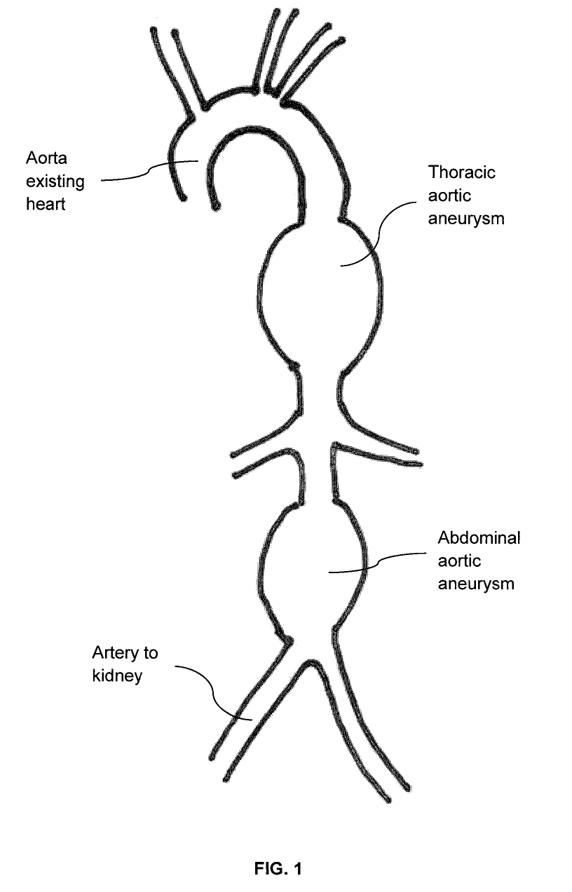

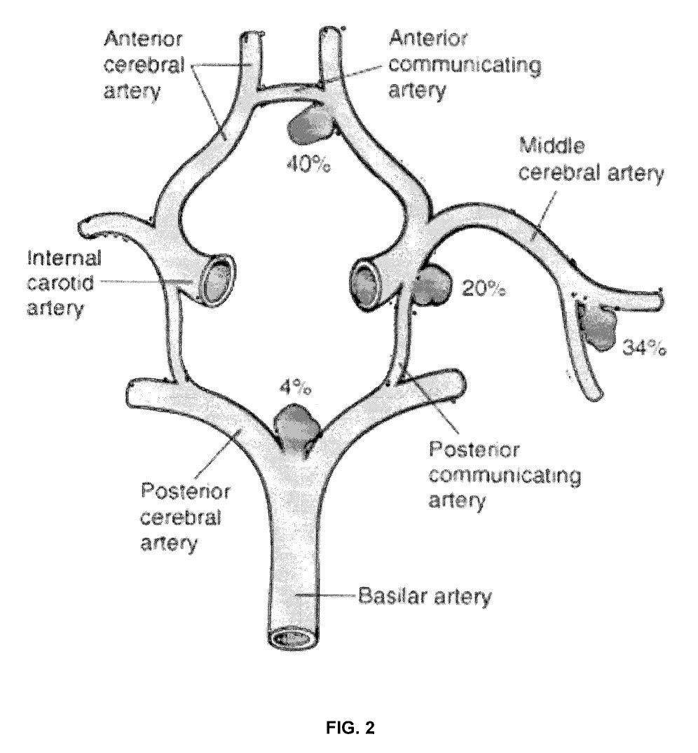

For purposes of brevity and clarity, descriptions of embodiments of the present disclosure are directed to stent graft devices, in accordance with the drawings in FIG. 1 to FIG. 13D. While aspects of the present disclosure will be described in conjunction with the embodiments provided herein, it will be understood that they are not intended to limit the present disclosure to these embodiments. On the contrary, the present disclosure is intended to cover alternatives, modifications and equivalents to the embodiments described herein, which are included within the scope of the present disclosure as defined by the appended claims. Furthermore, in the following detailed description, specific details are set forth in order to provide a thorough understanding of the present disclosure. However, it will be recognized by an individual having ordinary skill in the art, i.e. a skilled person, that the present disclosure may be practiced without specific details, and/or with multiple details arising from combinations of aspects of particular embodiments. In a number of instances, well-known systems, methods, procedures, and components have not been described in detail as not to unnecessarily obscure aspects of the embodiments of the present disclosure.

The measurement term “porosity” or “porosity percentage” used herein is in negative correlation with the term “coverage” or “coverage percentage”, which equals to (100−porosity) %, of a given surface area or an area on the membrane disclosed. Both terms “porosity percentage” and “coverage percentage” may be used interchangeably throughout the specification to indicate permeability of the disclosed embodiments. For example, a coverage of 15% means a porosity of 85%, and a porosity of 25% means a coverage of 75%.

In a representative or exemplary embodiment of the present disclosure, a stent graft device, as well as an endovascular repair kit comprising the stent graft device, is described hereinafter.

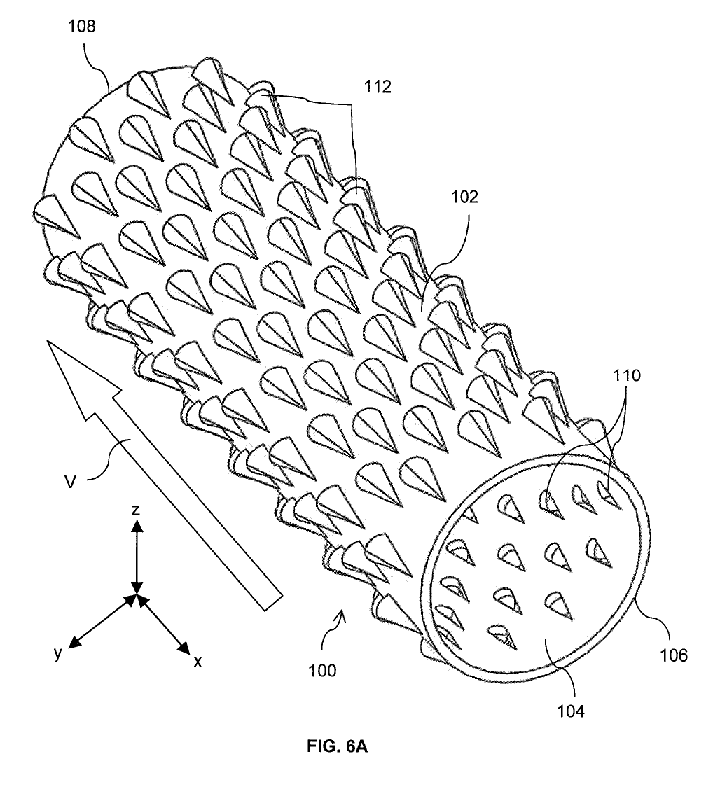

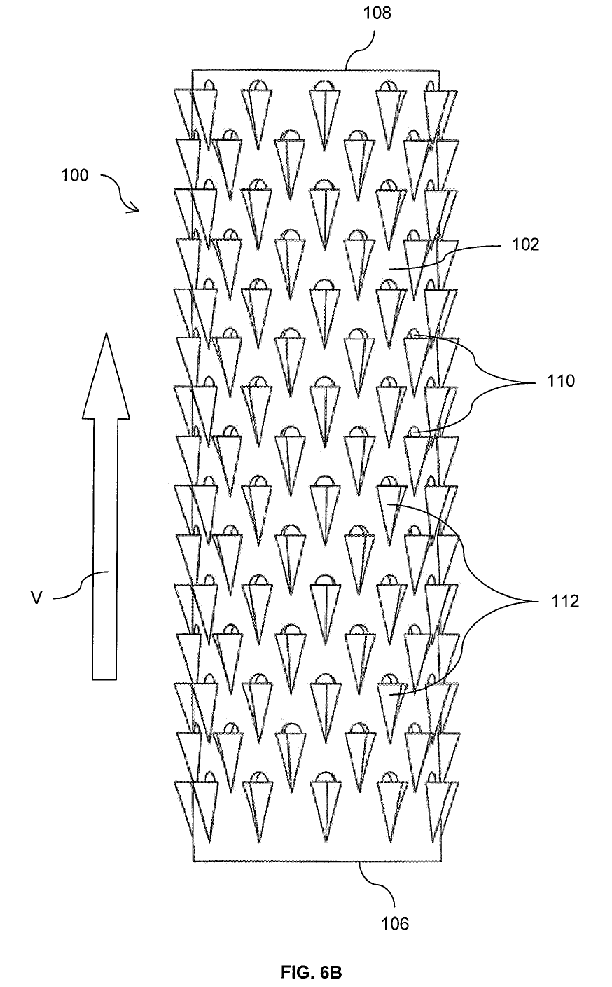

A stent graft device 100 of the representative embodiment is shown in FIG. 6A and FIG. 6B. The stent graft device 100 comprises a membrane 102 defining a lumen 104 between a proximal end 106 and a distal end 108 of the membrane 102. The stent graft device 100 is configured for implantation or insertion into an artery 200 and allows fluid communication (or blood flow) through the lumen 104, between the proximal end 106 and the distal end 108. Accordingly, the lumen 104 of the stent graft device 100 is configured for fluid communication distally therethrough. The stent graft device 100 further comprises a plurality of fenestrations 110 disposed on the membrane 102, and the plurality of fenestrations 110 are fluidly communicable with the lumen 104. The stent graft device 100 further comprises a plurality of protrusions 112 carried by the membrane 102. Each protrusion 112 extends/protrudes inwardly into the lumen 104 or outwardly from the lumen 104. As fluid is communicated (or blood is flowing) distally through the lumen 104 along the direction or directional vector V parallel to the x-axis, the fluid is communicated to the plurality of fenestrations 110. Fluid communicated from the plurality of fenestrations 110, e.g. fluid discharged out therefrom, is deflectable by the plurality of protrusions 112. Alternatively or additionally, fluid communicated from the plurality of fenestrations 110 is deflected by the plurality of protrusions 112, i.e. the plurality of protrusions 112 deflects fluid communicating through the lumen 104 outwardly through the plurality of fenestrations 110.

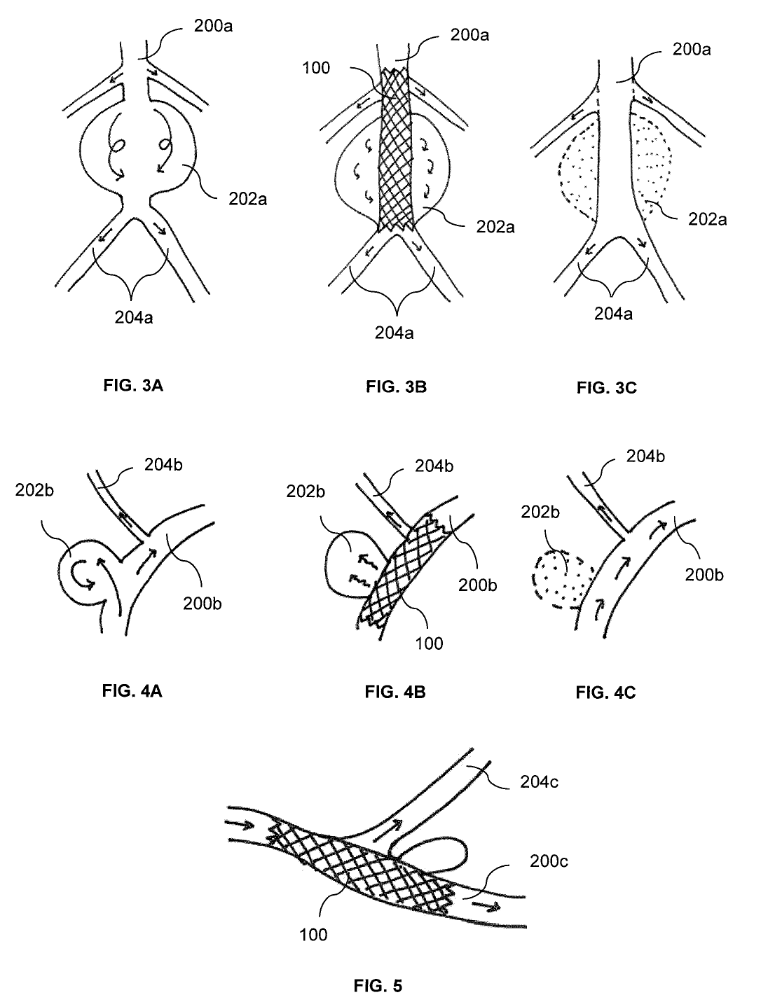

Thus, the stent graft device 100, particularly the membrane 102, is porous or partially permeable. When implanted or inserted into the artery 200, the stent graft device 100 does not function or work on the principle of excluding the aneurysm sac 202 from blood circulation. Instead, as demonstrated in FIG. 3A to FIG. 3C and FIG. 4A to FIG. 4C, the stent graft device 100 functions or works on the principle of flow deflection/diversion or partial flow deflection/diversion. The stent graft device 100 implanted or inserted into the artery 200 can be used to treat at least one or substantially all of the types of aneurysms occurring in the aorta. The stent graft device 100 can be used routinely regardless of whether or not the aneurysm involves the visceral vessels. Advantageously, the stent graft device 100 does not require customization for particular types of aortic aneurysms.

FIG. 3A to FIG. 3C are illustrations of an aorta 200a with aortic aneurysm before and after implantation of the stent graft device 100. FIG. 3A illustrates an aortic aneurysm before implantation of the stent graft device 100 wherein a pulsatile flow within the aneurysm sac 202a leads to growth and eventual rupture. FIG. 3B illustrates an aortic aneurysm immediately after implantation of the stent graft device 100 wherein the flow into the branch arteries/vessels 204a is maintained and there is turbulent flow within the aneurysm sac 202a. FIG. 3C illustrates an aortic aneurysm after implantation of the stent graft device 100 wherein eventually the aneurysm sac 202a occludes, the aortic vessel/aorta 200a remodels, and the flow into the branch arteries/vessels 204a is maintained.

FIG. 4A to FIG. 4C are illustrations of an artery 200b with saccular aneurysm before and after implantation of the stent graft device 100. FIG. 4A illustrates a saccular aneurysm before implantation of the stent graft device 100 wherein a pulsatile flow within the aneurysm sac 202b leads to growth and eventual rupture. FIG. 4B illustrates a saccular aneurysm immediately after implantation of the stent graft device 100 wherein the flow into the branch arteries/vessels 204b is maintained and there is turbulent flow within the aneurysm sac 202b. FIG. 4C illustrates a saccular aneurysm after implantation of the stent graft device 100 wherein eventually the aneurysm sac 202b occludes, the vessel/artery 200b remodels, and the flow into the branch arteries/vessels 204b is maintained.

The stent graft device 100 comprises the partially permeable membrane 102. As illustrated in FIG. 3B and FIG. 4B, the partially permeable stent graft device 100 can diffuse fluid communication or blood flow entering the aneurysm sac 202a,b, thereby resulting in fluid turbulence within the aneurysm sac 202a,b. As illustrated in FIG. 3C and FIG. 4C, such turbulence within the aneurysm sac 202a,b can lead to intra-aneurysmal thrombosis, which subsequently can lead to aneurysm occlusion and finally regression. At the same time, as illustrated in FIG. 3B, FIG. 3C, FIG. 4B, and FIG. 4C, flow into the branching arteries/vessels 204a,b (e.g. extracerebral arteries, abdominal visceral arteries, or renal arteries) can be maintained and can be prevented from being blocked. FIG. 5 illustrates the positioning of the stent graft device 100 across a representative carotid artery 200c with a branching artery/vessel 204c.

Referring to FIG. 6A and FIG. 6B, there is a stent graft device 100 having a plurality of protrusions 112 carried by a membrane 102. The stent graft device 100 further includes a plurality of fenestrations 110 that provides for porosity of the stent graft device 100. In the representative embodiment, the stent graft device 100 is tubular in shape and the protrusions 112 are generally triangular in shape. It would be readily apparent to the skilled person that other shapes are possible for implantation or insertion into an artery 200.

The stent graft device 100 thus has a tubular structure defining a channel or a lumen 104, configured to be coaxially aligned with an artery/vessel 200 such as the aorta for blood to flow through thereby. Particularly, the lumen 104 is configured for enabling fluid communication distally therethrough along a longitudinal axis of the lumen 104. The longitudinal axis of the lumen 104 is parallel to the x-axis. A plurality of protrusions 112, e.g. hollow teeth-like members, are fabricated on or carried by the external membrane 102 of the stent graft device 100. The membrane 102 and the lumen 104 extend between a proximal end 106 and a distal end 108 of the stent graft device 100. The proximal end 106 functions as an inlet and the distal end 108 functions as an outlet for fluid communication/blood flow through the lumen 104. Fluid communication through the lumen 104 is subsequently communicated out or discharged from the plurality of fenestrations 110 on the membrane 102. Fluid communicated from the plurality of fenestrations 110 is deflectable or deflected by the plurality of protrusions 112 radially away from a longitudinal axis of the lumen 104.

In one example, for protrusions 112 that extend inwardly into the lumen 104, fluid communicating through the lumen 104 engages at least one (inward) protrusion 112 before reaching a fenestration 110. The engagement with (inward) protrusion 112 enables the fluid to be deflected radially away from the longitudinal axis of the lumen 104. Thus, as the fluid is discharged from the fenestration 110, the fluid communicates through the fenestration 110 in a direction that is radially away from the longitudinal axis of the lumen 104. In another example, for protrusions 112 that extend outwardly from the lumen 104, fluid communicating through the lumen 104 engages at least one (outward) protrusion 112 after being discharged from a fenestration 110, wherein the (outward) protrusion 112 is positioned or disposed distal to the fenestration 110. The engagement with (outward) protrusion 112 enables the fluid to be deflected radially away from the longitudinal axis of the lumen 104. Thus, after the fluid is discharged from the fenestration 110, the fluid communicates along the surface of the membrane 102 until it engages the (outward) protrusion 112, thereby deflecting the fluid toward a direction is radially away from the longitudinal axis of the lumen 104.

In the representative embodiment, there are approximately 60 to 180 protrusions 112 carried by the membrane 102, depending on the sizes of the fenestrations 110 and/or membrane 102. Each protrusion 112 is disposed on the membrane 102 and extends outwardly from the lumen 104. Each protrusion 112 is or includes a raised structure/protuberance. Each fenestration or opening 110 is associated with or corresponds to a protrusion 112. Each fenestration 110 is disposed underneath and proximate to the protrusion 112. Viewed perpendicularly downward on the protrusion 112, e.g. as viewed from a plane normal to the z-axis, the protrusion 112 shields or covers a portion of the fenestration 110. Specifically, the protrusion 112 covers at least half of the fenestration 110. More specifically, the protrusions 112 collectively covers 50% to 98% (preferably 60% to 90%) of the fenestrations 110, resulting in a collective porosity of 2% to 50% (preferably 10% to 40%) for the stent graft device 100. In the representative embodiment, each protrusion 112 collectively covers 84% of a fenestration 110, resulting in a collective porosity of 16%.

In some other embodiments, the stent graft device 100 may additionally or alternatively comprise protrusions 112 that extend inwardly into the lumen 104. The above description regarding configurations of the collective or overall porosity/coverage of the membrane 102 apply analogously for protrusions 112 that extend inwardly into the lumen 104, e.g. recesses or depressions.

The plurality of fenestrations 110 can exhibit shapes such as, but not limited to, teardrop, ellipsoidal, or triangular shapes, or shapes generally geometrically similar to or correlated therewith. Each fenestration 110 has a pointed tip arranged to face against the incoming blood flow (e.g. toward or partially toward the proximal end 106), and an expanded end, edge, or tip 114 directed toward or partially toward the distal end 108 of the stent graft device 100. The expanded end 114 of each fenestration 110 slightly precedes or extends beyond the corresponding protrusion 112 as shown in FIG. 6B, while the rest of each fenestration 110 is covered/shielded by the corresponding protrusion 112. Each protrusion 112 thus gradually extends from the membrane 102 radially and/or distally away from a fenestration 110 for partially covering the fenestration 110.

Each protrusion 112 has a gradient 116 that enables the deflection of fluid communication radially away from the longitudinal axis. Each protrusion 112 can adopt an oblique formation, inclining downwards from an apex 118 and towards the proximal end 106 (or inclining upwards from a base 120 and towards the distal end 108), thereby forming the gradient 116. The gradients 116 of the protrusions 112 can have an angle α of 7 to 70 degrees with respect to the immediate underneath fenestration 110 or a planar surface of the membrane 102 or a plane normal to the z-axis (the plane being directly underneath the gradients 116). Specifically, the gradients 116 can have an angle α of 15 to 45 degrees. More specifically, in the representative embodiment, the gradients 116 have an angle α of 27 degrees.

Each protrusion 112 can be further crosswise (along the y-axis) arched to create the apex 118. The arching of the protrusions 112, e.g. the magnitude or height of concavity/convexity, may be varied, such as to be 1 mm or less. The protrusions 112 can be formed in different shapes or profiles, for instance, as illustrated in FIG. 8A to FIG. 8C. The shapes of the protrusions 112 can be semi-circular, elongated, tubular and/or conical. Other shapes are also possible. The stent graft device 100 can thus have the protrusions 112 in one or more of the shapes illustrated in FIG. 8A to FIG. 8C. The protrusions 112 can be manufactured, fabricated, and/or integrated with the membrane 102 of the stent graft device 100 and extended radially outward away from the longitudinal axis of the lumen 104, such that lumen 104 is smooth and the membrane 102 is textured.

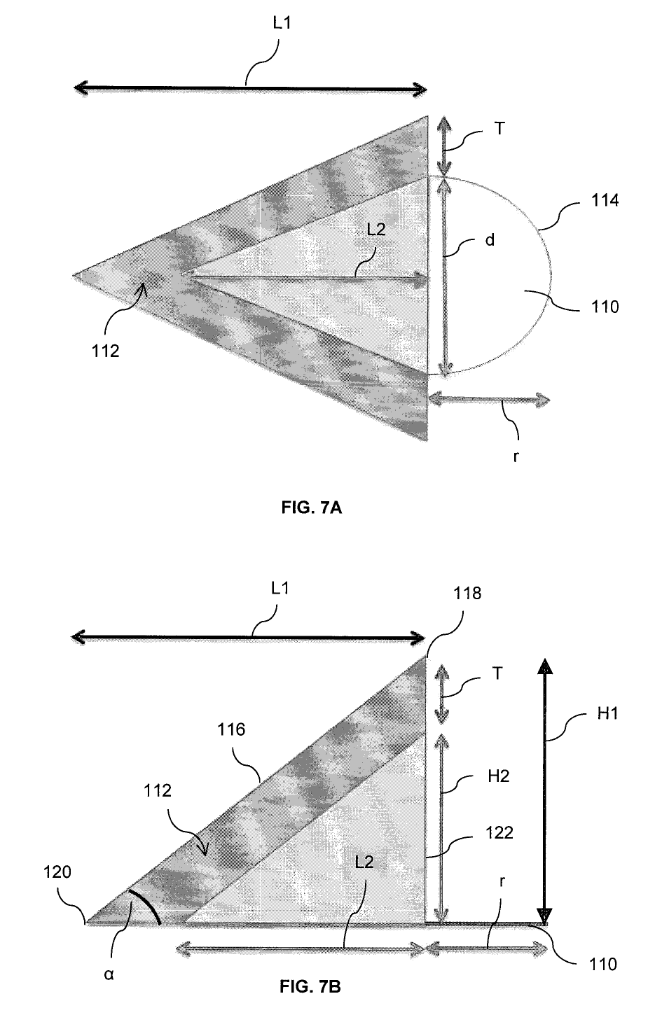

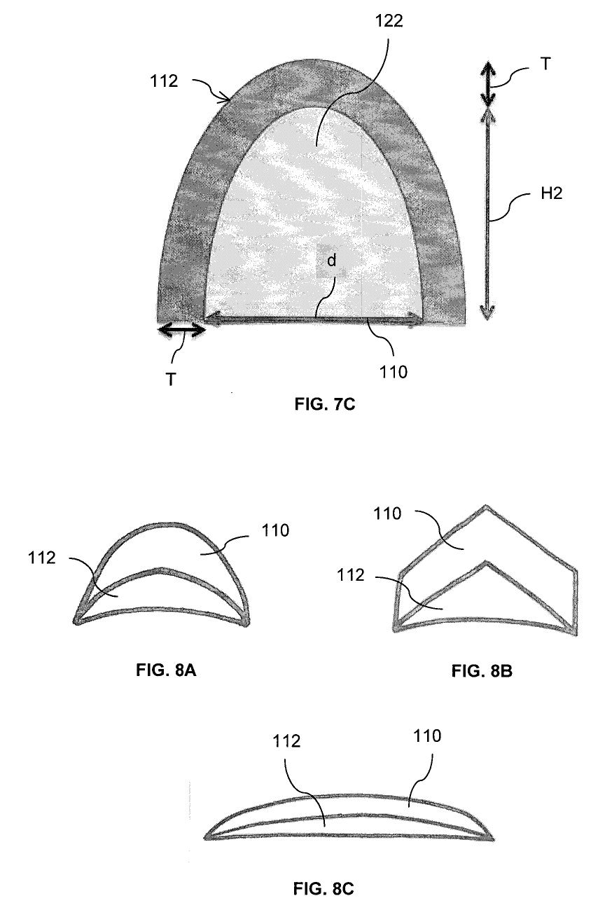

In the representative embodiment, each protrusion 112 has a triangular shape or profile, as illustrated in FIG. 7A to FIG. 7C. In a top or planar view as shown in FIG. 7A (viewed from a plane normal to the z-axis), the fenestration 110 has a diameter d (or radius r) and is partially covered by the protrusion 112 of a corresponding protrusion 112. The protrusion 112 has an outer length L1 and an inner length L2, resulting in a wall thickness T. In a side or lateral view as shown in FIG. 7B (viewed from a plane normal to the y-axis), the protrusion 112 has an inner height H2. As the protrusion 112 has a uniform wall thickness T throughout, the overall height or outer height H1 of the protrusion 112 is formed by the combination of the inner height H2 and the wall thickness T. In a front view as shown in FIG. 7C (viewed from a plane normal to the x-axis), the protrusion 112 arches across the fenestration 110 and forms an opening 122 that is fluidly communicable with the corresponding fenestration 110. The opening 122 faces, or at least partially faces, the distal end 108 of the stent graft device 100.

The diameter d of a fenestration 100 can range from 1 mm to 5 mm (2 mm in the representative embodiment), and correspondingly the radius r can range from 0.5 mm to 2.5 mm (1 mm in the representative embodiment). The outer length L1 of the protrusion 112 can range from 2 mm to 8 mm (6.1 mm in the representative embodiment). The inner length L2 is 4 mm in the representative embodiment. The outer height of the protrusion 112 can range from 1 mm to 5 mm (4.7 mm in the representative embodiment). The inner height H2 in the representative embodiment is 4 mm. The wall thickness of the protrusion 112 is 0.7 mm.

In several embodiments of the present disclosure, the dimensions of the fenestrations 110 and the protrusions 112 range between a minimum and a maximum, with a preferred value for the representative embodiment. The table below summarizes the dimensions of the fenestrations 110 and the protrusions 112.

| | | | Representative |
|---|---|---|---|
| Dimension | Minimum | Maximum | Embodiment |
| d | 1 mm | 5 mm | 2 mm |
| r | 0.5 mm | 2.5 mm | 1 mm |
| L1 | 2 mm | 8 mm | 6.1 mm |
| L2 | — | — | 4 mm |
| H1 | 1 mm | 5 mm | 4.7 mm |
| H2 | — | — | 4 mm |
| T | — | — | 0.7 mm |
| α | 7° | 70° | 27° |
| Coverage | 50% | 98% | 84% |
| Porosity | 50% | 2% | 16% |

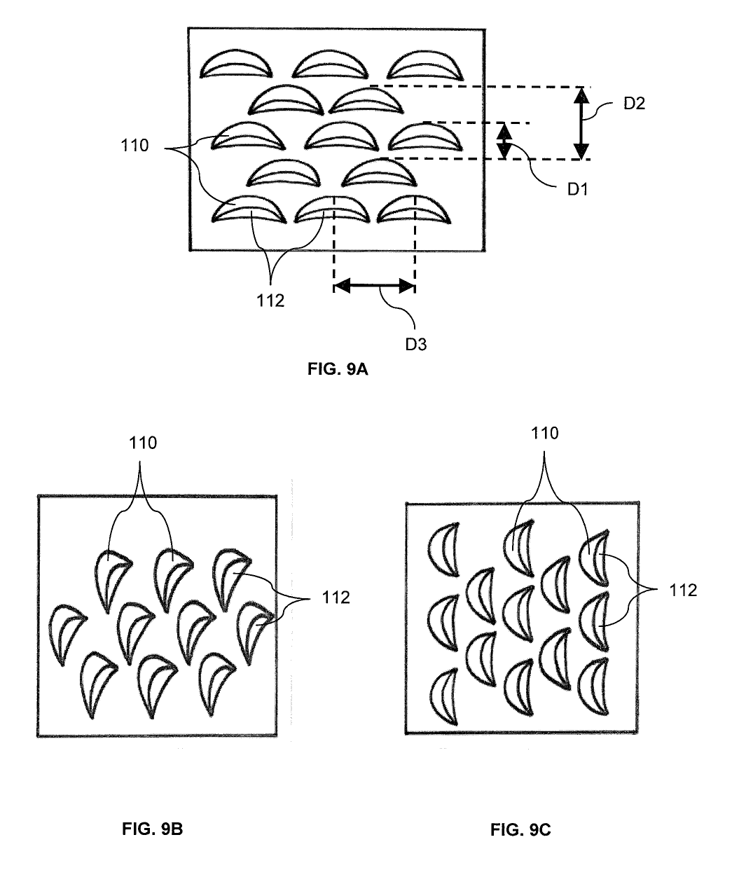

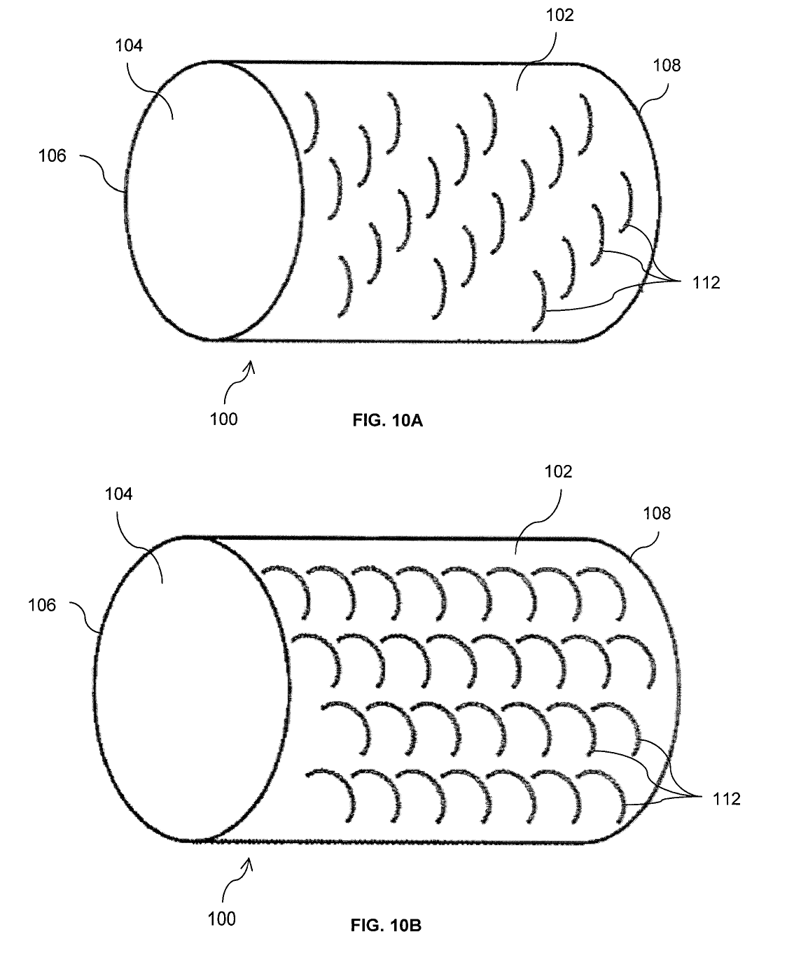

With reference to FIG. 6A and FIG. 6B, in the representative embodiment, the plurality of protrusions 112 are tapered lengthwise towards the proximal end 106 or inlet of the lumen 104 of the stent graft device 100. The protrusions 112 can be uniformly disposed, and equidistantly spaced, in longitudinal and transverse/lateral orientations relative to the tubular structure of the stent graft device 100. Accordingly, a plurality of radially arranged rows of protrusions 112 is formed on the membrane 102 of the stent graft device 100. Moreover, as shown in FIG. 6B and FIG. 9A, a protrusion 112 of a given row can be positioned in a manner that is offset between two neighbouring protrusions 112 within proximally and distally adjacent rows of protrusions 112. In general, the protrusions 112 can be positioned relative to one another such that fluid communication or blood flow is directed through the fenestrations 110. Typically, the blood stream flows towards longitudinally aligned protrusions 112, where such longitudinally aligned protrusions 112 are disposed in alternate transversely aligned rows of protrusions 112. Upon reaching a protrusion 112, the oblique and tapered profile (due to the gradient 116) of the protrusion 112 progressively deflects the blood stream outward (e.g. radially away from the longitudinal axis of the lumen 104) to produce a radial blood flow pattern or direction relative to the longitudinal axis.

Referring to the arrangement of the plurality of protrusions 112 shown in FIG. 9A, the distance D1 between two adjacent rows of longitudinally non-overlapping/offset protrusions 112 can be approximately 2 mm to 10 mm, preferably 5 mm to 10 mm. The distance D2 between successive rows of longitudinally overlapping protrusions 112 can be approximately 3 mm to 15 mm, preferably 7 mm to 15 mm. Further, the distance D3 in between two lateral or transverse neighbouring protrusions 112 is approximately 2 mm to 10 mm, preferably 5 mm to 10 mm. In the representative embodiment, the distance D1 is 7 mm, the distance D2 is 10 mm, and the distance D3 is 7 mm.

The arrangement and/or positioning of the protrusions 112 can be varied along the longitudinal and/or circumferential directions of the membrane 102 and/or stent graft device 100, wherein turbulence is promoted in some areas/regions by producing vortices, and laminar flow is promoted in other areas/regions. In some embodiments, the outer height H1 of the protrusions 112 does not significantly increase the height profile of the membrane 102 and/or stent graft device 100 during delivery because it is possible to limit the outer height H2 of the protrusions 112, such as to a maximum height limit of 1 mm or lower.

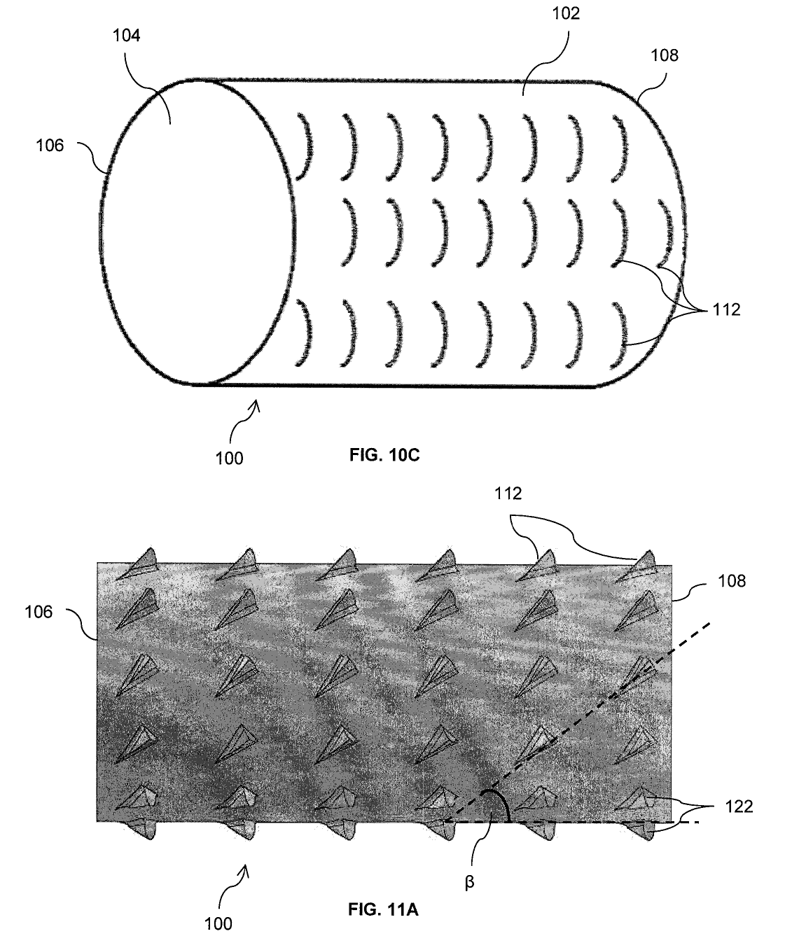

In some alternative embodiments, other arrangements of the plurality of protrusions 112 carried by the membrane 102 are possible, as would be apparent to a skilled person. FIG. 9B and FIG. 9C illustrates examples of alternative arrangements of the protrusions 112. In FIG. 9B, the protrusions 112 are tilted approximately 45 degrees, with respect to the longitudinal axis, away from the distal end 108. In FIG. 9C, the protrusions 112 are tilted approximately 90 degrees, with respect to the longitudinal axis, away from the distal end 108. FIG. 10A to FIG. 10C illustrates additional examples of arrangements of the plurality of protrusions 112 carried by the membrane 102.

Thus, at least one or some of the protrusions 112 can be tilted to face a different direction or to face partially toward the distal end 108, in order to alter or the flow dynamics relative to the membrane 102 of the stent graft device 100. The protrusions 112 have the purpose of affecting not only the re-direction of the flow relative to the membrane 104, to either be more laminar or more turbulent, but also reducing the drag on the membrane 104 of the stent graft device 100, thus, reducing the risk of stent graft migration after implantation. For instance, the openings 122 of the protrusions 112 can be arranged to form a lateral angle in relation to the blood stream flowing through the lumen 104. The gradients 116 of each protrusion 112 are directed along vectors at least partially toward the distal end 108 of the stent graft device 100. Accordingly, the openings 122 formed by each protrusion 112 are at least partially facing the distal end 108 of the stent graft device 100, depending on the lateral angle. Referring to FIG. 6A and FIG. 6B, the openings 122 of the protrusions 112 are all facing directly toward the distal end 108. However, in some alternatively embodiments, the gradients 116 of at least two protrusions 112 are directed along different vectors. In other words, there are at least two protrusions 112 with openings 122 that face different directions toward or partially toward the distal end 108.

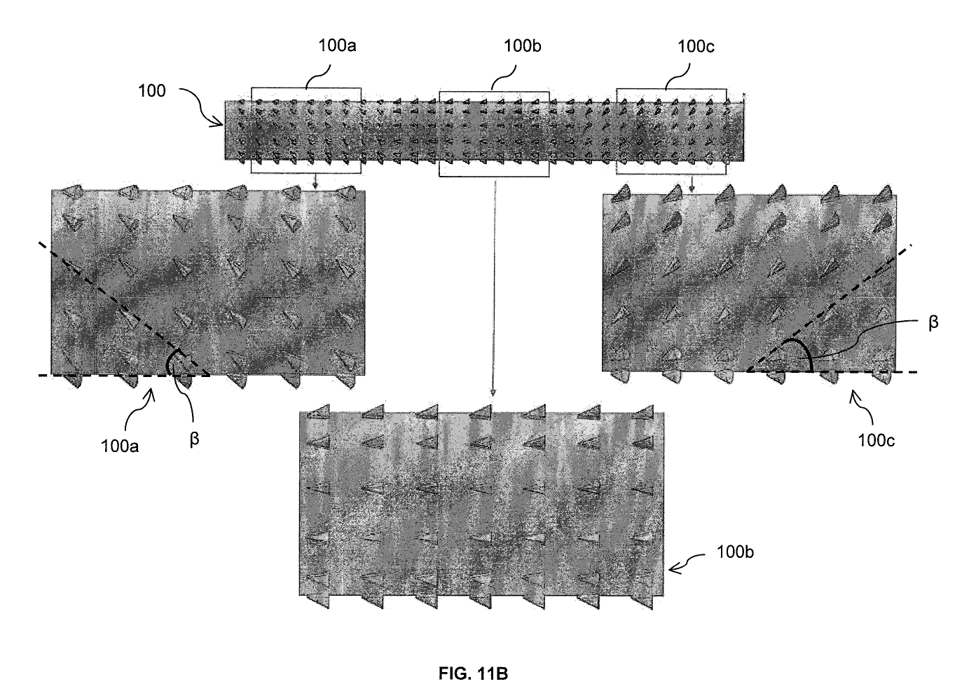

FIG. 11A illustrates an alternative embodiment of the stent graft device 100, wherein the plurality of protrusions 112 are tilted at a lateral angle β with respect to a sectional plane longitudinally through a protrusion 112 (the plane being normal to the y-axis). Accordingly, the lateral angle β is transverse to the angle α. The lateral angle β may theoretically range from 0 to 90 degrees, but preferably between 30 to 60 degrees. In the arrangement shown in FIG. 11A, each protrusion 112 has a lateral angle β of 45 degrees.

In another alternative embodiment as shown in FIG. 11B, the stent graft device 100 has a plurality of protrusions 112 that are tilted at a lateral angle β. However, not all of the protrusions 112 are tilted at the same lateral angle β. In a proximal section 100a of the stent graft device 100, the protrusions 112 therein are tilted at a lateral angle β of 45 degrees rightwards from the plane normal to the y-axis. In a middle section 100b of the stent graft device 100, the protrusions 112 therein are not tilted, i.e. the protrusions 112 are tilted at a lateral angle β of 0 degrees. In a distal section 100c of the stent graft device 100, the protrusions 112 therein are tilted at a lateral angle β of 45 degrees leftwards from the plane normal to the y-axis. Thus, the plurality of protrusions 112 of the stent graft device 100 are facing in three distinct directions, toward or partially toward the distal end 108. Other arrangements and configurations of the directions and lateral angles β of the protrusions 112 are possible, as readily understood by the skilled person. For example, the lateral angles β of the protrusions 112 may enable the facing directions of the openings 122 of the protrusions 112 to form a serpentine pattern, or even a spiral pattern, from the proximal end 106 to the distal end 108.

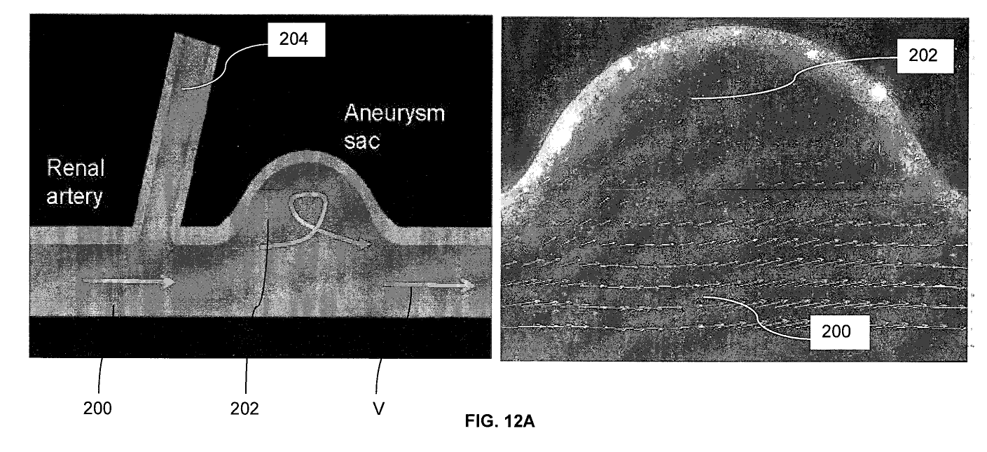

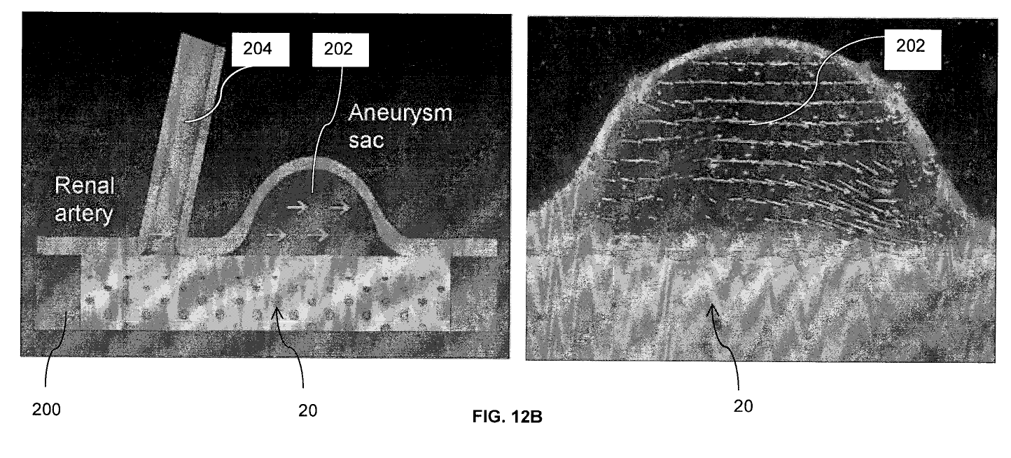

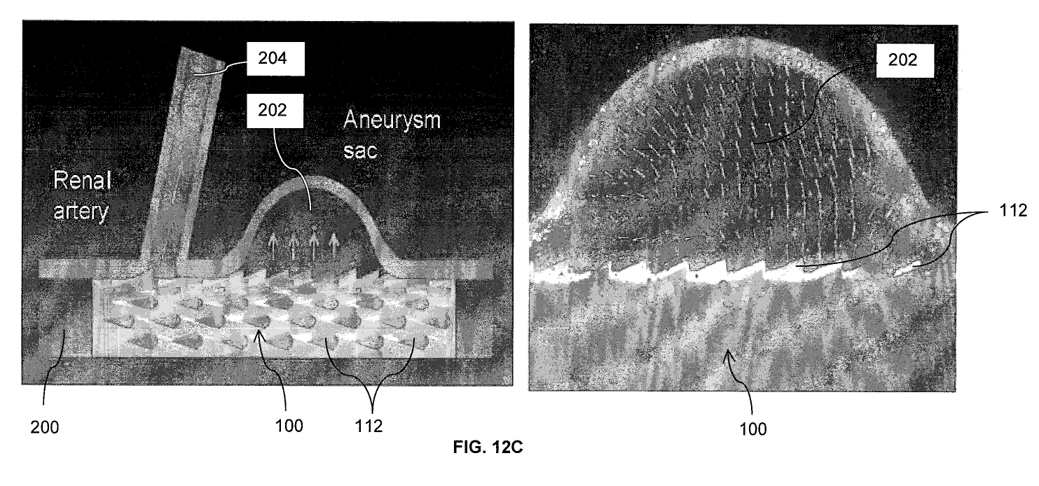

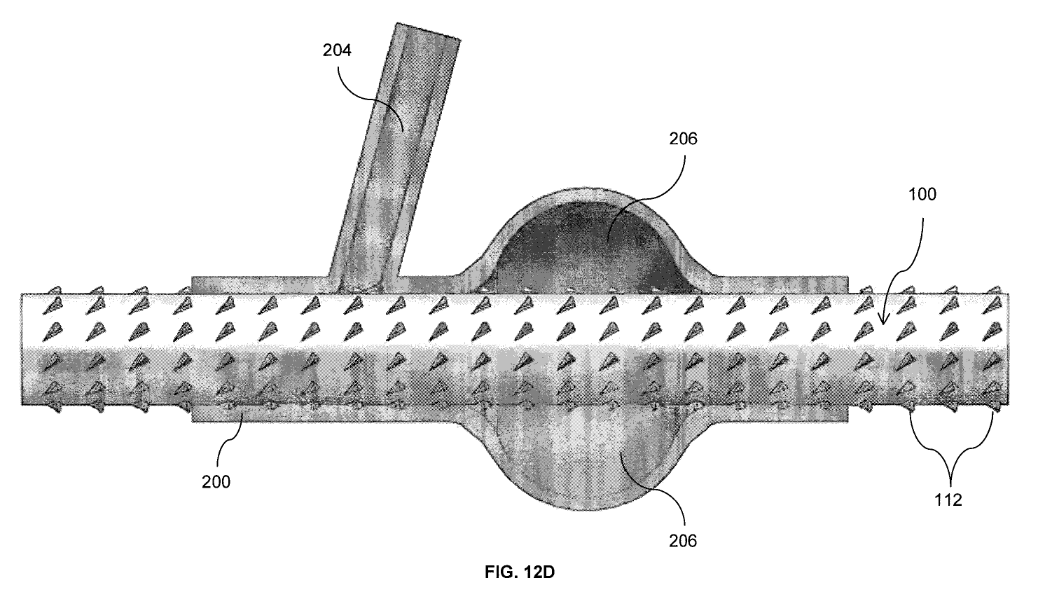

With reference to FIG. 12C, upon insertion or implantation into an artery 200, fluid communication or blood flow through the artery 200 also communicates or flows through the lumen 104 of the stent graft device 100. The stent graft device 100 comprises the plurality of protrusions 112 and the plurality of fenestrations 110, wherein the protrusions 112 extend outwardly from the membrane 102 to partially cover/shield the fenestrations 110 immediately underneath. Accordingly, a protrusion 112 is paired or corresponds with a fenestration 110. As the fluid communication/blood flow is in the distal direction, the protrusion 112 substantially impedes fluid communication/blood flow proximally from the fenestration 110. As the protrusion 112 partially covers the fenestration 110, the protrusion 112 partially impedes fluid communication/blood flow radially from the fenestration 112. This partial impedance by the protrusion 112 also deflects fluid/blood discharged from the fenestration 112 at least partially distally toward another protrusion 112. Further, fluid/blood discharged out from the fenestrations 110 may flow laminarly along the membrane 102 until it reaches and engages another protrusion disposed distally thereto, wherein the other protrusion causes deflection of the fluid communication/blood flow radially away from the longitudinal axis of the lumen 104. The plurality of protrusions 112 of the stent graft device 100 thus offers better control over the flow dynamics of the incoming fluid communication/blood flow.

In some alternative embodiments, the plurality of protrusions 112 extends inwardly into the lumen 104. Such “inwardly” protrusions 112 include extensions from the inner surface of the membrane 102, and portions of the membrane 102 that are depressed or recessed inwards into the lumen 104, thereby forming depressions or recesses. This allows the outer surface of the membrane 102 to be smooth and more easily inserted/implanted into the artery 200. During fluid communication/blood flow through the lumen 104, there is engagement of the fluid/blood with the protrusions 112 that extend inwardly into the lumen 104. The engagement enables the fluid/blood to be deflected radially away from the longitudinal axis of the lumen 104. Thus, as the fluid/blood is discharged from the fenestration 110, the fluid/blood communicates or flows through the fenestration 110 in a direction that is radially or partially radially away from the longitudinal axis of the lumen 104. In some other alternative embodiments, the stent graft device 100 may comprise a plurality of protrusions 112 wherein some protrusions 112 are extending outwardly from the lumen 104 and other protrusions 112 are extending inwardly into the lumen 104.

The plurality of fenestrations 110 are accessible to (e.g. fluidly communicable with) the lumen 104 such that a portion of the fluid communication/blood flow passing thereby is directed into an aneurysm sac 202, along or in an intended direction relative to the aneurysm sac 202. The fenestrations 110 permit fluid communication/blood flow between the lumen 104 and the aneurysm sac 202. Moreover, the arrangement of the plurality of protrusions 112 carried by the membrane 102 is such that fluid communication/blood flow is directed through the fenestrations 110 and into the aneurysm sac 202.

For instance, blood flowing into the aneurysm sac 202 is in a radial flow pattern or direction as illustrated in FIG. 12C. The radial flow of the blood stream generated in the aneurysm sac 202 can be further attributed to and tailored by a tapered slope or gradients 116 formed on the protrusions 112. Such radial flow of the blood stream diverted into the aneurysm sac 202 distributes the fluid pressure substantially evenly onto, across, or within the aneurysm sac 202, thereby reducing the fluid distal flow velocity (i.e. velocity of fluid flow distally along the longitudinal axis of the lumen 104). Blood entering the aneurysm sac 202 without any stent graft device or with a conventional stent graft device 20, as respectively shown in FIG. 12A and FIG. 12B, tends to guide the blood to flow in a substantially laminar direction such that the distal side of the aneurysm sac 202 generally receives or is exposed to fluid communicating at a high fluid distal flow velocity, and consequently a much greater fluid pressure. This imbalance in fluid pressure on the aneurysm sac 202 results an elevated risk of further expansion and/or rupture of the aneurysm sac 202. In FIG. 12A which shows blood flow in the artery 200 without any stent graft devices, there is a vortex flow pattern within the aneurysm sac 202. In FIG. 12B which shows blood flow in the artery 200 with a conventional stent graft device 20, e.g. a commercial flow diverter, implanted, there is a forward or distal direction flow pattern within the aneurysm sac 202.

In each of FIG. 12A to FIG. 12C, the figure on the left is an illustration of the artery 200 inserted with, or without, a stent graft device, and the figure on the right is a respective image of flow dynamics based on particle imaging velocimetry. The table below summarizes how the implantation of the stent graft device 100, as well as a conventional stent graft device 20, can affect the fluid dynamics of blood flow in the aneurysm sac 202.

| | | Conventional stent | |
|---|---|---|---|
| | Without | graft device 20/ | Stent graft device |
| | any stent | Commercial flow | 100 of the present |
| Design | graft device | diverter | disclosure |
| Fluid distal | 0.096 m/s | 0.014 m/s | 0.006 m/s |
| flow velocity | | | |
| Fluid distal | — | 85% | 94% |
| flow reduction | | | |

In order to have blood flowing into the aneurysm sac 202 in an intended manner, the distances between the protrusions 112 on the membrane 102, i.e. the arrangement of the protrusions 112, cannot be too close to or too far apart from one another, as this can result in less desirable blood flow dynamics relative to the aneurysm sac 202. The implantation of the stent graft device 100 can affect blood flow into the aneurysm sac 202 by substantially reducing the fluid distal flow velocity in the aneurysm sac 202. The lower fluid flow velocity in the distal direction reduces the fluid pressure at the distal side of the aneurysm sac 202, thereby mitigating the risks of further expansion and/or rupture of the aneurysm sac 202, which is caused due to extensive blood flow into the aneurysm sac 202. Advantageously, the stent graft device 100 facilitates localized thrombosis in the aneurysm sac 202, which can lead to relining of the arterial wall of that particular vessel segment, and subsequently to the occlusion and regression of the aneurysm sac 202.

In some embodiments, the artery 200 is implanted with the stent graft device 100 as shown in FIG. 11A. An illustration of such an embodiment is shown in FIG. 12D, wherein the artery 200 has a fusiform aneurysm 206. The lateral angle β of the protrusions 112 shifts the direction of fluid communication/blood flow laterally away from the longitudinal axis of the lumen 104. Thus, the velocity of fluid flowing distally along the longitudinal axis is reduced due to the resolution of velocity vectors. This would further reduce fluid flow velocity in the distal direction and the fluid pressure at the distal side of the fusiform aneurysm 206, thereby further mitigating the risks of further expansion and/or rupture of the fusiform aneurysm 206. Experimental data indicates that having a positive lateral angle β for the protrusions 112 can improve the probability of causing thrombosis in the fusiform aneurysm 206.

In the representative embodiment, the stent graft device 100 is directly implantable into the artery 200 for treating endovascular aneurysms. The stent graft device 100 may be expandable from a crimped/compressed/collapsed state, thereby allowing for easier implantation into the artery 200 in its crimped state. In some other embodiments, the stent graft device 100 may further comprise a stent coaxially disposed within the lumen 104, and fluidly communicable with the membrane 102 and the lumen 104. The additional stent or stent platform is tubular and can provide structural support to the stent graft device 100, especially when implanted into the artery 200. The membrane 102 of the stent graft device 100 can be positioned anywhere along the stent that provides longitudinal and radial strength, support, and/or fixation to prevent migration of the stent graft device 100 within the artery/vessel 200.

Examples of a stent include a mesh stent, web stent, ring stent, or intravascular stent. The stent or stent platform can be expandable from a crimped/compressed/collapsed state. For example, the stent may be self-expandable or balloon expandable. Alternatively, the stent can be a hybrid having self-expandable and balloon expandable features. The stent can be made from stainless steel (e.g. grade 316L), cobalt chromium, nickel titanium (nitinol), or any combination. Other materials are also possible, as readily understood by the skilled person. The stent may be marked with radiopaque markers to assist in its positioning within the stent graft device 100 and within the artery 200.

The stent graft device 100 can be used for treating endovascular aneurysms (e.g. saccular aneurysms, fusiform aneurysms, and dissecting aneurysms) that can occur within any artery/vein/vessel 200 in the body. The stent graft device 100 can be used to treat at least one or substantially all of the types of aneurysms. For example, the stent graft device 100 can be used to treat intravascular aneurysms occurring in blood vessels in the cerebral region, carotid region, peripheral region, and/or aortic region. The stent graft device 100 can also be used to treat saccular aneurysms, fusiform aneurysms, and/or dissecting aneurysms.

The stent graft device 100, particularly the membrane 102, comprises at least one longitudinal and/or radial strip or stripe of material and/or fabric, wherein the longitudinal/radial strip(s)/stripe(s) comprises the plurality of fenestrations 110 and the plurality of protrusions 112. Alternatively or additionally, the membrane 102 comprises a mesh material and/or fabric, wherein the mesh material/fabric comprises the plurality of fenestrations 110 and the plurality of protrusions 112.

Further alternatively or additionally, the membrane 102 comprises a woven material and/or fabric (e.g. a weave), wherein the woven material/fabric comprises the plurality of fenestrations 110 and the plurality of protrusions 112. The stent graft device 100 can be made from expanded PTFE, Dacron, polyester, polyurethane, silicone, or any combination thereof. Other materials are also possible, as readily understood by the skilled person. The porosity (or coverage area) of the stent graft device 100, specifically the membrane 102, can be uniform or can vary across the longitudinal direction (x-axis) and/or radial direction. The membrane 102 may be marked with radiopaque markers to assist in its positioning within the stent graft device 100 and within the artery 200.

According to the second aspect of the present disclosure, in various representative embodiments, there is an endovascular repair kit comprising the stent graft device 100 described above, together with a stent delivery system.

The stent graft device 100 can be incorporated within the stent delivery system, which can be repositionable and/or retrievable. Generally, the delivery system is composed of two different sub-systems, namely a deployment sub-system and an introducer system. In the deployment system, there is an inner catheter where the stent graft device 100 is temporarily and coaxially placed in the inner catheter. In the introducer sub-system, there is an outer catheter telescopically covering part of the inner catheter including the stent graft device 100. The stent graft device 100 is preferably in a crimped/compressed/collapsed state prior to its implantation/insertion/deposition into the artery/vessel 200.

The coaxially joined outer and inner catheters are then inserted into the subject to reach the treatment site. At the artery/vessel 200 to be treated, the outer catheter is progressively retracted to expose the stent graft device 100, specifically the membrane 102, around the inner catheter. The stent graft device 100, in the crimped state, can be re-positioned before being triggered into the expanded state for anchoring onto the artery/vessel 200 via the stent deployment mechanism carried on the deployment sub-system. Similar actions can be performed again by practitioners to deploy a second or even third stent graft device 100.

In embodiments of the endovascular repair kit wherein the stent graft device 100 comprises the stent, the stent delivery system may deliver the stent graft device 100, specifically the membrane 102 and stent, in a separate and sequential manner.

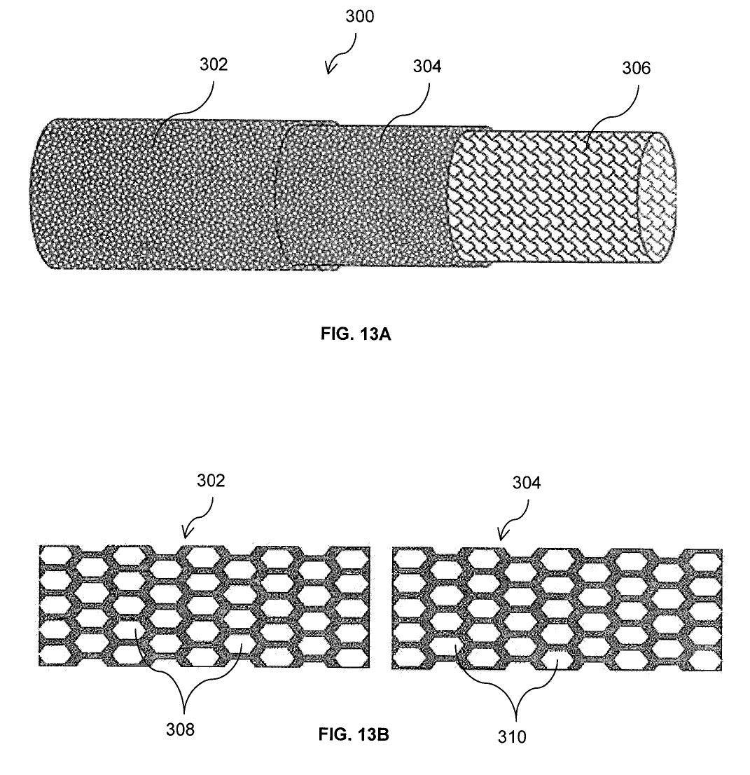

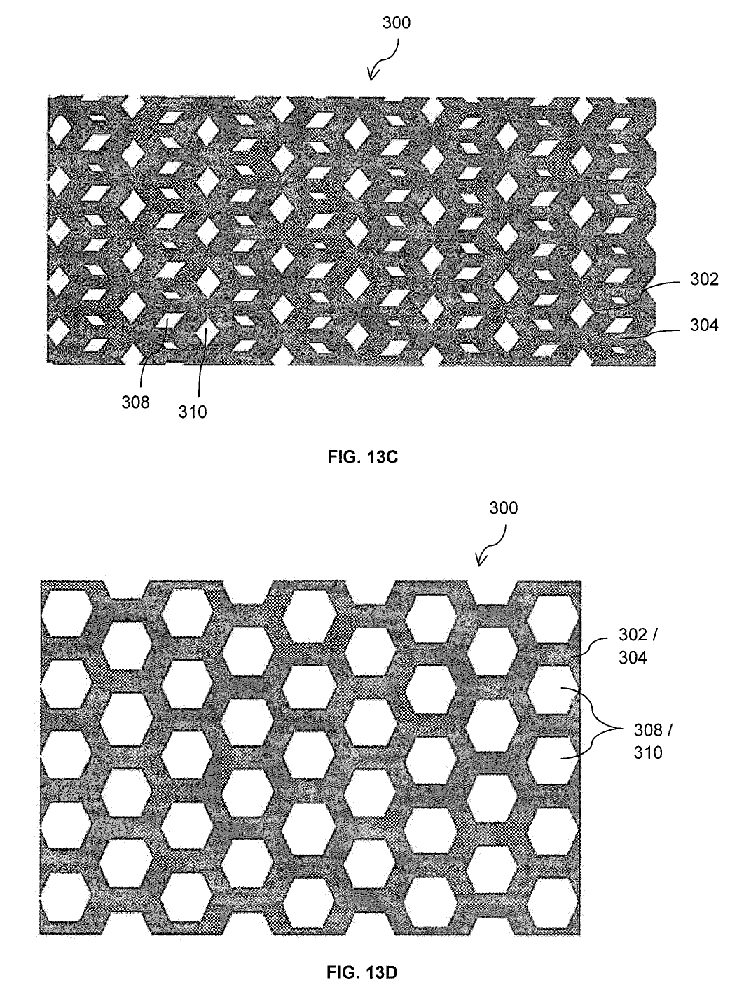

According to another aspect of the present disclosure, in several embodiments, there is a stent graft device 300 with adjustable porosity. An illustration of the stent graft device 300 is shown in FIG. 13A. The stent graft device 300 is implantable or insertable into an artery/vessel 200 for treating endovascular aneurysms by diverting fluid communication/blood flow in the artery/vessel 200.

The stent graft device 300 comprises an outer membrane 302 which is porous or partially permeable, and an inner membrane 304 which is also porous or partially permeable. The outer membrane 302 and the inner membrane 304 are concentric and/or coaxial with respect to each other. The outer membrane 302 and the inner membrane 304 are overlapped (e.g. the outer membrane 302 can overlap the inner membrane 304). The outer membrane 302 comprises an outer surface and an inner surface. The inner membrane 304 also comprises an outer surface and an inner surface. As part of an implantation procedure, the concentric and/or coaxial outer membrane 302 and inner membrane 304 can be moved or slid along each other along a common longitudinal axis to alter the overall porosity of the stent graft device 300. For example, the outer surface of the inner membrane 304 can be slid along, under and/or against the inner surface of the outer membrane 302 to adjust the overall porosity. The inner surface of the outer membrane 302 can be slid along, over and/or against the outer surface of the inner membrane 304 to adjust the overall porosity. Preferably, the outer membrane 302 and the inner membrane 304 are coaxially positioned relative to each other in a fashion to provide collective porosity percentage of 2% to 50% or coverage percentage of 50% to 98% to the stent graft device 300.

The outer membrane 302 and the inner membrane 304 exhibit a tubular structure, giving the stent graft device 300 a tubular shape or profile. The stent graft device 300 is directly implantable into the artery 200 for treating endovascular aneurysms. The stent graft device 300 may be expandable from a crimped/compressed/collapsed state, thereby allowing for easier implantation into the artery 200 in its crimped state. In some other embodiments, the stent graft device 300 may further comprise a stent 306 disposed concentric and/or coaxial with respect to the outer membrane 302 and the inner membrane 304, and fluidly communicable therewith. The additional stent 306 or stent platform is tubular and can provide structural support to the stent graft device 300, especially when implanted into the artery 200. Examples of a stent 306 include a mesh stent, web stent, ring stent, or intravascular stent. The stent 306 or stent platform can be expandable from a crimped/compressed/collapsed state. For example, the stent 306 may be self-expandable or balloon expandable. Alternatively, the stent 306 can be a hybrid having self-expandable and balloon expandable features. The stent 306 can be made from stainless steel (e.g. grade 316L), cobalt chromium, nickel titanium (nitinol), or any combination. Other materials are also possible, as readily understood by the skilled person. At least one of the outer membrane 302, inner membrane 304, and stent 306 may be marked with radiopaque markers to assist in the positioning of each component within the stent graft device 300 and within the artery 200.

The inner membrane 304 can be positioned anywhere along the stent 306 that provides both longitudinal and radial strength to prevent migration of the stent graft device 300. The stent 306 can be positioned and/or located under the inner membrane 304 (i.e. under the concentric and/or coaxial inner membrane 304 and outer membrane 302). The stent 306 can provide longitudinal and radial strength, support, and/or fixation.

The positions of the outer membrane 302, inner membrane 304, and/or stent 306 can be adjusted with respect to one another to provide sufficient coverage across an aneurysm sac 202 to induce intra-aneurysmal thrombosis. Their positions can also be adjusted with respect to one another to provide sufficient porosity across the aortic branches and/or visceral arteries 204 to maintain blood flow into these arteries.

FIG. 13B illustrates a portion of the stent graft device 300, wherein the outer membrane 302 and the inner membrane 304 are separated from each other. FIG. 13C illustrates a manner in which the concentric and/or coaxial outer membrane 302 and inner membrane 304 can be overlapped such that the overlapping can result in minimum porosity and maximum coverage. FIG. 13D illustrates a manner in which the concentric and/or coaxial outer membrane 302 and inner membrane 304 can be overlapped such that the overlapping can result in maximum porosity and minimum coverage.

Referring to FIG. 13B, the outer membrane 302 comprises a plurality of fenestrations/pores/openings/holes 308 and the inner membrane 304 comprises a plurality of fenestrations/pores/openings/holes 310. Each of the outer membrane 302 and the inner membrane 304 has an outer diameter of about 18 mm to 25 mm. Each of the outer membrane 302 and the inner membrane 304 can have a relatively thinner thickness of about 0.15 mm to 0.30 mm, or a relatively thicker thickness of about 0.5 mm to 1 mm. One of the outer membrane 302 and the inner membrane 304 can be fabricated to be thicker/thinner than the other. Each fenestration 308/310 is fabricated onto the respective membrane 302/304, and generally has a diameter of 3 mm to 5 mm. The fenestrations 308 and 310 can be fabricated in a configuration similar to, but not necessarily identical to, honeycomb structures as shown in FIG. 13B.

In some embodiments, the outer membrane 302 is fabricated to be less permeable than the inner membrane 304. The relative permeability or porosity or fenestrations/pores coverage of the outer membrane 302 is about 30% to 60%. The relative permeability or porosity or fenestrations/pores coverage of the inner membrane 304 is about 60% to 90%.

Selective positioning of the outer membrane 302 and the inner membrane 304 relative each other results in one or more overall effective fenestration sizes, thus adjusting the collective or overall porosity/coverage of the stent graft device 300. This is due to parts of the structure of the outer membrane 302 or inner membrane 304 overlapping the fenestrations 310 and 308, respectively. An example of such an arrangement is shown in FIG. 13C, wherein the stent graft device 300 has maximum coverage and minimum porosity. Alternatively, the plurality of fenestrations 308 of the outer membrane 302 and the plurality of fenestrations 310 of the inner membrane 304 have substantially similar or identical areas. The fenestrations 308 and 310 can be aligned to be substantially or almost coincident with and overlapping one another. An example of such an arrangement is shown in FIG. 13D, wherein the stent graft device 300 has minimum coverage and maximum porosity.

The outer membrane 302 and inner membrane 304 comprises at least one longitudinal and/or radial strip or stripe of material and/or fabric, wherein the longitudinal/radial strip(s)/stripe(s) comprises the plurality of fenestrations 308 and 310, respectively. Alternatively or additionally, the outer membrane 302 and inner membrane 304 comprises a mesh material and/or fabric, wherein the mesh material/fabric comprises the plurality of fenestrations 308 and 310. Further alternatively or additionally, the membrane 102 comprises a woven material and/or fabric (e.g. a weave), wherein the woven material/fabric comprises a plurality of fenestrations 308 and 310. The plurality of fenestrations 308 and 310 can have one or more types of shapes. The plurality of fenestrations 308 and 310 may be disposed on the same plane, or extend/project/protrude radially outwards and/or inwards.

The stent graft device 300 can be made from expanded PTFE, Dacron, polyester, polyurethane, silicone, or any combination thereof. Other materials are also possible, as readily understood by the skilled person. The porosity (or coverage area) of the stent graft device 300, specifically the outer membrane 302 and inner membrane 304, can be uniform or can vary across the longitudinal direction (x-axis) and/or radial direction. The porosity/coverage of the outer membrane 302 and the porosity/coverage of the inner membrane 304 can be substantially similar or the same. Alternatively, they can be dissimilar or different.

According to another aspect of the present disclosure, in various representative embodiments, there is an endovascular repair kit comprising the stent graft device 300 described above, together with a stent delivery system.

The stent graft device 300 can be incorporated within the stent delivery system, which can be repositionable and/or retrievable. The stent graft device 300 can be implanted into the artery/vessel 200 using the stent delivery system, which can be a conventional or a low profile stent delivery system. The outer membrane 302, inner membrane 304, and stent 306 can be delivered separately and sequentially, thereby allowing for a smaller delivery profile. Additionally, the separate and sequential delivery of the outer membrane 302 and inner membrane 304 can allow for the ends of the stent 306 to be uncovered or expanded to provide better anchorage within the artery/vessel 200. The stent graft device 300 can be mounted onto the stent delivery system (e.g. a low profile stent delivery system) having a distal end and a proximal end, wherein the stent 306 can be positioned at the distal end of the stent delivery system, and wherein the inner membrane 302 is positioned at the proximal end of the stent delivery system. Once positioned across the aneurysm sac 202, the stent 306 can be partially deployed across the neck or entrance of the aneurysm sac 202 by the stent delivery system followed by the deployment of the inner membrane 304 that goes above, over, and/or around the stent 306. Referring to FIG. 13C and FIG. 13D, the overall porosity/coverage of the stent graft device 300 can be adjusted and/or modified by adjusting the position of the inner membrane 304 and the outer membrane 302 with respect to each other.

One or more stent delivery systems can be used to deliver the stent graft device 300 in a separate and/or sequential manner, where the inner membrane 304 is first delivered by a first stent delivery system across the neck of the aneurysm sac 202 and keeping it in place. The outer membrane 302 is subsequently delivered, preferably via a second stent delivery system, by sliding over the inner membrane 304. The stent 306 is then fully expanded over the inner membrane 304 and outer membrane 302 to secure both in position. The first and second stent delivery systems can refer to a single integrated system being used in a sequential fashion to deposit the membranes 302 and 304.

In embodiments of the endovascular repair kit wherein the stent graft device 300 comprises the stent 306, the stent delivery system may deliver the stent graft device 300, specifically the outer membrane 302, inner membrane 304, and stent 306, in a separate and sequential manner. Further, the inner membrane 304 may be delivered to the site to be treated through a first stent delivery system followed by the delivery of the outer membrane 302 using a second stent delivery system.

It will be readily apparent to and understood by a skilled person that the aforementioned stent delivery system(s) described for the stent graft device 300 may be analogously and similarly applicable for the stent graft device 100 in another aspect of the present disclosure.

The stent graft device 300 can be used for treating endovascular aneurysms (e.g. saccular aneurysms, fusiform aneurysms, and dissecting aneurysms) that can occur within any artery/vein/vessel 200 in the body. The stent graft device 300 can be used to treat at least one or substantially all of the types of aneurysms. For example, the stent graft device 300 can be used to treat intravascular aneurysms occurring in blood vessels in the cerebral region, carotid region, peripheral region, and/or aortic region. The stent graft device 300 can also be used to treat saccular aneurysms, fusiform aneurysms, and/or dissecting aneurysms.

Various stent graft devices have been disclosed in several embodiments and aspects of the present disclosure for treating aneurysms. Endovascular aortic repair (EVAR) using stent graft devices have become the standard of care for the treatment of aortic aneurysms. Numerous stent graft devices for routine use have been developed but only five stent grafts have approval from the United States Food and Drug Administration (FDA) and are commercially available in the United States. Up to 45% of patients are unsuitable for EVAR using routine stent graft devices as they have a short proximal graft landing zone (e.g. less than 15 mm of normal aorta from the nearest visceral vessel). Stent graft device manufacturers have attempted to overcome this shortcoming and other disadvantages by offering customized stent graft devices with fenestrations to accommodate the visceral vessels. However, these customized stent graft devices are costly, require specialized training with a high level of technical competence to utilize, and require 6 to 10 weeks to manufacture. As such, customized stent graft devices are not easily available and cannot be used in emergent and/or emergency situations (e.g. sudden rupture of the aneurysm or aneurysm sac). Another limitation of existing or conventional stent graft devices is the large delivery profile and limited flexibility of the stent delivery system. As such, patients with small-sized access or tortuous vessels (femoral or iliac arteries) are also unsuitable for EVAR.

The partially permeable stent graft devices 100 and 300 of the present disclosure can overcome, avoid and/or at least ameliorate one or more of the limitations of currently available stent graft devices. Firstly, the flow diversion design of the stent graft device 100/300 allows the visceral blood flow to be maintained while causing thrombosis of the aneurysm sac 202. As such, the stent graft device 100/300 can be deployed across visceral arteries without compromising distal flow along the artery 200. This makes the stent graft device 100/300 ideal for aneurysms with short landing zones (e.g. a short neck) when defined by conventional criteria as the stent graft device 100/300 extend the landing zone to include non-aneurysmal good quality aorta. More importantly, the stent graft device 100/300 can be used in aneurysms where open surgery is associated with high morbidity/mortality (e.g. thoracoabdominal or aortic arch aneurysms).

Additionally, by separating the delivery of the stent graft device 100/300 and the delivery of the stent, the delivery profile of the stent graft device 100/300 is significantly reduced. The reduced delivery profile allows the stent graft device 100/300 to be used in applications involving patients with small-sized or tortuous access vessels. The design of the stent graft device 100/300 also obviates the requirement of practitioners to have specialized training to utilize the stent graft device 100/300. Further, the stent graft device 100/300 is designed such that there is no need for customized manufacturing to meet certain criteria for individual patients/subjects. As such, the stent graft device 100/300 can be used by any practitioner or vascular surgeon with endovascular surgery skills in emergent and/or emergency situations for treating the aneurysms described above and throughout the present disclosure.

In the foregoing detailed description, embodiments of the present disclosure in relation to stent graft devices are described with reference to the provided figures. The description of the various embodiments herein is not intended to call out or be limited only to specific or particular representations of the present disclosure, but merely to illustrate non-limiting examples of the present disclosure. The present disclosure serves to address at least some of the mentioned problems and issues associated with the prior art. Although only some embodiments of the present disclosure are disclosed herein, it will be apparent to a person having ordinary skill in the art in view of this disclosure that a variety of changes and/or modifications can be made to the disclosed embodiments without departing from the scope of the present disclosure. The scope of the disclosure as well as the scope of the following claims is not limited to embodiments described herein.

1. A stent graft device comprising:
a membrane defining a lumen between a proximal end and a distal end of the membrane, the lumen for fluid communication distally therethrough;
a plurality of fenestrations disposed on the membrane and fluidly communicable with the lumen; and
a plurality of protrusions carried by the membrane, each protrusion extending inwardly into or outwardly from the lumen,
wherein fluid communicated from the plurality of fenestrations is deflectable or deflected by the plurality of protrusions.

2. The stent graft device as in claim 1, wherein fluid communicated from the plurality of fenestrations is deflectable or deflected by the plurality of protrusions radially away from a longitudinal axis of the lumen.

3. The stent graft device as in claim 2, wherein fluid communicated from the plurality of fenestrations is deflected by protrusions extending inwardly into the lumen.

4. The stent graft device as in claim 2, wherein fluid communicated from the plurality of fenestrations is deflectable by protrusions extending outwardly from the lumen.

5. The stent graft device as in claim 4, wherein fluid communicated from a fenestration is deflectable by at least one protrusion disposed distal to the fenestration.

6. The stent graft device as in claim 4, wherein each protrusion has a gradient for deflection of fluid communication radially away from the longitudinal axis.

7. The stent graft device as in claim 6, wherein a protrusion gradually extends from the membrane radially and/or distally away from a fenestration, partially covering the fenestration.

8. The stent graft device as in claim 7 wherein the protrusion defines an opening fluidly communicable with the fenestration, the opening at least partially facing the distal end.

9. The stent graft device as in claim 7, wherein the protrusion substantially impedes fluid communication proximally from the fenestration.

10. The stent graft device as in claim 7, wherein the fenestration is at least half covered by the protrusion.

11. The stent graft device as in claim 7, wherein the protrusion partially impedes fluid communication radially from the fenestration.

12. The stent graft device as in claim 11, wherein fluid communication from the fenestration toward the protrusion is deflectable by the protrusion at least partially distally toward another protrusion.

13. The stent graft device as in claim 6, wherein the gradient of each protrusion is directed along vectors at least partially toward the distal end.

14. The stent graft device as in claim 13, wherein the gradients of at least two protrusions are directed along different vectors.

15. The stent graft device as in claim 1, further comprising a stent coaxially disposed within the lumen.

16. The stent graft device as in claim 15, wherein the stent is expandable from a crimped state.

17. An endovascular repair kit comprising:
a stent delivery system; and
a stent graft device comprising:
a membrane defining a lumen between a proximal end and a distal end of the membrane, the lumen for fluid communication distally therethrough;
a stent coaxially disposed within the lumen;
a plurality of fenestrations disposed on the membrane and fluidly communicable with the lumen; and
a plurality of protrusions carried by the membrane, each protrusion extending inwardly into or outwardly from the lumen,
wherein fluid communicated from the plurality of fenestrations is deflectable or deflected by the plurality of protrusions.

18. The endovascular repair kit as in claim 17, wherein the stent delivery system is repositionable and/or retrievable.
